(19) United States
(12) Patent Application Publication
(10) Pub. No.: US 2025/0064408 A1
(43) Pub. Date: Feb. 27, 2025

(54) ON-DEMAND TRANSMISSION OF ANALYTE DATA

(71) Applicant: Dexcom, Inc., San Diego, CA (US)

(72) Inventors: Michael A. PLOOF, San Diego, CA (US); Lorenzo Gorospe SUBIDO, San Diego, CA (US); Gang CHEN, San Diego, CA (US); Natalie Carolyn RIPCHICK, San Diego, CA (US); David Lee LEMKE, Afton, MN (US)

(21) Appl. No.: 18/797,029

(22) Filed: Aug. 7, 2024

Related U.S. Application Data
(60) Provisional application No. 63/578,630, filed on Aug. 24, 2023.

(51) Int. Cl. A61B 5/00; A61B 5/145; G06F 3/04847; G06F 3/0488; G16H 40/67

(52) U.S. Cl. CPC A61B 5/7475; A61B 5/0004; A61B 5/002; A61B 5/145; A61B 5/7435; G06F 3/04847; G16H 40/67; A61B 2560/0209; G06F 3/0488

(57) ABSTRACT

Techniques for on-demand transmission of analyte data are disclosed. In certain embodiments, a system for on-demand transmission of analyte data includes an analyte sensor, at least one input sensor, and analyte sensor electronics circuitry. The analyte sensor electronics circuitry includes a connectivity interface. The analyte sensor electronics circuitry is configured to process touch input data received via the at least one input sensor to identify a first touch input pattern and, based on identifying the first touch input pattern, transition the connectivity interface from a low-power state to an operational state. The analyte sensor electronics circuitry is further configured to, upon transitioning the connectivity interface to the operational state, transmit first analyte data to a display device, and transition the connectivity interface from the operational state to the low-power state after transmitting the first analyte data to the display device.

### CROSS-REFERENCE TO RELATED APPLICATIONS

This application claims priority to and benefit of U.S. Provisional Patent Application No. 63/578,630, filed Aug. 24, 2023, which is assigned to the assignee hereof and hereby expressly incorporated herein in its entirety as if fully set forth below and for all applicable purposes.

### BACKGROUND

Diabetes is a metabolic condition relating to the production or use of insulin by the body. Insulin is a hormone that allows the body to use glucose for energy, or store glucose as fat.

Diabetes mellitus is a disorder in which the pancreas cannot create sufficient insulin (Type I or insulin dependent) and/or in which insulin is not effective (Type 2 or non-insulin dependent). In the diabetic state, the victim suffers from high blood sugar, which causes an array of physiological derangements (kidney failure, skin ulcers, or bleeding into the vitreous of the eye) associated with the deterioration of small blood vessels. A hypoglycemic reaction (low blood sugar) may be induced by an inadvertent overdose of insulin, or after a normal dose of insulin or glucose-lowering agent accompanied by extraordinary exercise or insufficient food intake.

Conventionally, a diabetic patient carries a self-monitoring blood glucose (SMBG) monitor, which may require uncomfortable finger pricking methods. Due to the lack of comfort and convenience, a diabetic will normally only measure his or her glucose level two to four times per day. Unfortunately, these time intervals are spread so far apart that the diabetic will likely be alerted to a hyperglycemic or hypoglycemic condition too late, sometimes incurring dangerous side effects as a result. In fact, it is unlikely that a diabetic will take a timely SMBG value, and further the diabetic will not know if his blood glucose value is going up (higher) or down (lower), due to limitations of conventional methods.

Consequently, a variety of non-invasive, transdermal (e.g., transcutaneous) and/or implantable sensors are being developed for continuously detecting and/or quantifying blood glucose values. Generally, in a diabetes management system, a transmitter associated with the sensor wirelessly transmits raw or minimally processed data for subsequent display and/or analysis at one or more display devices, which can include a mobile device, a server, or any other type of communication devices. A display device, such as a mobile device, may then utilize a trusted software application (e.g., approved and/or provided by the manufacturer of the sensor), which takes the raw or minimally processed data and provides the user with information about the user's blood glucose levels. Because diabetes management systems using such implantable sensors can provide more up-to-date information to users, they may reduce the risk of a user failing to regulate the user's blood glucose levels.

This background is provided to introduce a brief context for the summary and detailed description that follow. This background is not intended to be an aid in determining the scope of the claimed subject matter nor be viewed as limiting the claimed subject matter to implementations that solve any or all of the disadvantages or problems presented above.

### SUMMARY

Certain embodiments provide a system for on-demand transmission of analyte data, the system including an analyte sensor, at least one input sensor, and analyte sensor electronics circuitry. The analyte sensor electronics circuitry includes a connectivity interface. The analyte sensor electronics circuitry is configured to process touch input data received via the at least one input sensor to identify a first touch input pattern and, based on identifying the first touch input pattern, transition the connectivity interface from a low-power state to an operational state. The analyte sensor electronics circuitry is further configured to, upon transitioning the connectivity interface to the operational state, transmit first analyte data to a display device, and transition the connectivity interface from the operational state to the low-power state after transmitting the first analyte data to the display device.

Certain embodiments provide a system for on-demand transmission of analyte data. The system includes an analyte sensor and analyte sensor electronics circuitry. The analyte sensor electronics circuitry includes a connectivity interface. The analyte sensor electronics circuitry is configured to process one or more radiofrequency (RF) signals received from a display device via the connectivity interface to identify a first wakeup pattern and, based on identifying the first wakeup pattern, transition the connectivity interface from a low-power state to an operational state. The analyte sensor electronics circuitry is further configured to, upon transitioning the connectivity interface to the operational state, transmit first analyte data to the display device, and transition the connectivity interface from the operational state to the low-power state after transmitting the first analyte data to the display device.

### BRIEF DESCRIPTION OF THE DRAWINGS

FIG. 1A depicts a health management system that may be used in connection with certain embodiments of the present disclosure.

FIG. 1B illustrates the example health management system of FIG. 1A in more detail, according to certain embodiments disclosed herein.

FIG. 2A illustrates a perspective view of an outer housing of the analyte sensor system of FIG. 1A, according to certain embodiments disclosed herein.

FIG. 2B illustrates a bottom perspective view of the outer housing of the analyte sensor system of FIG. 2A, according to certain embodiments disclosed herein.

FIG. 3 illustrates a cross-sectional view of the outer housing of the analyte sensor system of FIG. 2A and FIG. 2B, according to certain embodiments disclosed herein.

FIG. 4 is a flow diagram illustrating example operations for triggering on-demand transmission of analyte data based on touch input, according to certain embodiments disclosed herein.

FIG. 5 is a flow diagram illustrating example operations for detecting a combination of touch input for false wake rejection, according to certain embodiments disclosed herein.

FIG. 6 is a flow diagram illustrating example operations for implementing a timeout period after waking the sensor electronics circuitry and/or connectivity interface of the analyte sensor system from a low-power state, according to certain embodiments disclosed herein.

FIG. 7 is a flow diagram illustrating example operations for rejecting false wakeup of the analyte sensor system based on orientation, according to certain embodiments disclosed herein.

FIG. 8 is a flow diagram illustrating example operations for on-demand transmission of analyte data based on proximity RF, according to certain embodiments disclosed herein.

### DETAILED DESCRIPTION

Analyte monitoring systems may include an analyte sensor system comprising an analyte senor (e.g., a glucose sensor) for measuring analyte (e.g., glucose) levels of a patient and a sensor electronics circuitry for processing analyte sensor data or information from the analyte sensor. The analyte sensor may communicate raw sensor measurements to the sensor electronics circuitry for processing. Thereafter, the analyte sensor system may transmit corresponding analyte values (e.g., glucose values or levels) and/or the raw data to a patient's display device, such as a mobile phone. In order to initially connect with the display device, the analyte sensor system is configured to transmit or broadcast one or more advertisement packets to the display device through one or more advertisement channels.

In response to the advertisement packets, the display device and the analyte sensor system may engage in a connection request/response exchange to establish a connection. Subsequently, the display device and the analyte sensor system may engage in authentication, pairing, and/or bonding. After bonding, the two devices exchange data (e.g., analyte values), and may then disconnect. Once the analyte sensor system and the display device have paired and bonded, at each of the devices, information about the other device and the bond that has been created with the other device is stored. For example, at the analyte sensor system, the display device is added to a “targeted device list,” where information about the bond that has been created with the display device is stored and then used for reconnections. As a result, pairing and bonding will not be necessary during reconnections.

In order to conserve power, the analyte sensor system may reconnect to the display device (e.g., to transmit updated analyte data to the display device) only periodically, such as every 5 minutes. For example, a connectivity interface (e.g., a BLE module) within the analyte sensor system may transmit analyte data (e.g., estimated glucose values or “EGVs”) to the display device and then enter a low-power state for a fixed period of time. Once the fixed period of time has elapsed, the connectivity interface may transition from the low-power state back into the operational state and reconnect to the display device, and the analyte sensor system may transmit additional analyte data to the display device via the connectivity interface. Once the additional analyte data has been transmitted, the connectivity interface (and/or one or more other components of the analyte sensor system) then re-enters the low-power state for the fixed period of time.

While periodically waking the connectivity interface from the low-power state to transmit analyte data to the display device extends the battery life of the analyte sensor system, a user may wish to view analyte data on the display device more frequently than the fixed transmission intervals would permit. For example, a user may want to view an EGV immediately after waking, before or after eating or drinking, before or after physical activity, or before going to sleep. In such situations, as a result of how existing analyte monitoring systems are configured, the user must wait for the fixed period of time to expire (e.g., up to 5 minutes) until updated analyte data is transmitted to the display device.

Accordingly, in various embodiments of the present disclosure, the analyte sensor system may transmit analyte data to the display device and/or perform other functions on-demand, instead of requiring the user to wait for a fixed period of time. For example, a user may trigger on-demand transmission of analyte data via touch input, such as by pressing and/or tapping on the analyte sensor system. In some embodiments, each unique pattern of presses and/or taps may trigger on-demand transmission of a different type of analyte data. In some embodiments, the analyte sensor system may implement a two-phase wakeup technique to distinguish between intentional user input requesting analyte data to be transmitted, and inadvertent input that causes a connectivity interface (e.g., a BLE module) of the analyte sensor system to frequently wake from the low-power state, which may decrease battery life. In addition, on-demand transmission of analyte data may be triggered via the display device (e.g., via a button on a touchscreen interface) by causing the display device to generate a radiofrequency (RF) signal pattern, such as an on/off keying (OOK) pattern that causes the connectivity interface in the analyte sensor system to wake from the low-power state. Accordingly, a user is able to obtain updated analyte data on-demand, without significantly impacting the battery life of the analyte sensor system.

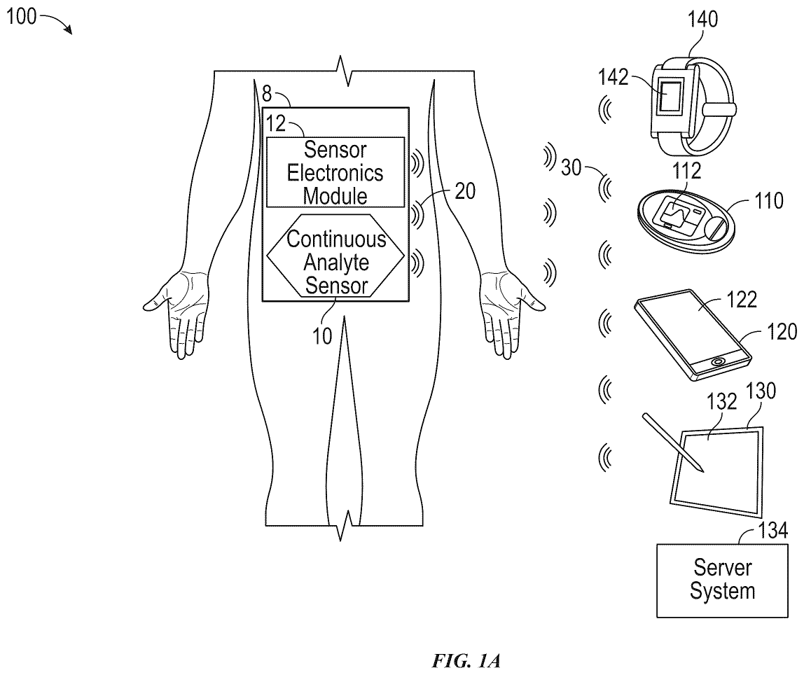

The above techniques for on-demand transmission of analyte data are described below in additional detail in conjunction with FIGS. 1A, 1B, 2A, 2B, and 3-8 below. It should be noted that, although certain embodiments herein are described with respect to the management of diabetes, a glucose sensor system, and the transmission of glucose measurements between the devices, the protocols and techniques described herein are similarly applicable to any type of health management system that includes any type of analyte sensor (e.g., lactate sensor, ketone sensor, O₂ sensor, etc.).

### Example Analyte Sensor System

FIG. 1A depicts a health management system 100 (“system 100”), such as a diabetes management system, that may be used in connection with certain embodiments of the present disclosure. Certain embodiments may involve triggering on-demand transmission of analyte data with the health management system 100. Health management system 100 depicts aspects of analyte sensor system 8 (hereinafter “SS 8”) that may be communicatively coupled to display devices 110, 120, 130, and 140, and/or to server system 134.

In certain embodiments, SS 8 is provided for measurement of an analyte in a host or a user. By way of an overview and an example, SS 8 may be implemented as an encapsulated microcontroller that makes sensor measurements, generates analyte data (e.g., by calculating values for continuous glucose monitoring data), and engages in wireless communications (e.g., via Bluetooth and/or other wireless protocols) to send such data to remote devices, such as display devices 110, 120, 130, 140 and/or server system 134. U.S. App. No. 2019/0336053, which is incorporated herein in its entirety by reference, further describes an on-skin sensor assembly that, in certain embodiments, may be used in connection with SS 8.

In certain embodiments, SS 8 includes sensor electronics circuitry 12 and an analyte sensor 10 associated with sensor electronics circuitry 12. In certain embodiments, sensor electronics circuitry 12 (also referred to herein as “analyte sensor electronics circuitry”) includes electronic circuitry associated with measuring and processing analyte sensor data or information, including algorithms associated with processing and/or calibration of the analyte sensor data/information. Sensor electronics circuitry 12 may be physically/mechanically connected to analyte sensor 10 and can be integral with (i.e., non-releasably attached to) or releasably attachable to analyte sensor 10.

Sensor electronics circuitry 12 may also be operatively coupled to analyte sensor 10, such that the components may be electromechanically coupled to one another (e.g., (a) prior to insertion into a patient's body, or (b) during the insertion into the patient's body). Sensor electronics circuitry 12 may include hardware, firmware, and/or software that enable measurement and/or estimation of levels of the analyte in a host/user via analyte sensor 10 (e.g., which may be/include a glucose sensor). For example, sensor electronics circuitry 12 can include one or more potentiostats, a power source for providing power to analyte sensor 10, other components useful for signal processing and data storage, and a telemetry module for transmitting data from the sensor electronics circuitry to one or more display devices. For example, SS 8 can wirelessly transmit 20 data to a display device 110, 120, 130, 140, and a display device 110, 120, 130, 140 can wirelessly transmit 30 data to SS 8. Electronics can be affixed to a printed circuit board (PCB) within SS 8, or platform or the like, and can take a variety of forms. For example, the electronics can take the form of an integrated circuit (IC), such as an Application-Specific Integrated Circuit (ASIC), a microcontroller, a processor, and/or a state machine.

Sensor electronics circuitry 12 may include sensor electronics that are configured to process sensor information, such as sensor data, and generate transformed sensor data and displayable sensor information. Examples of systems and methods for processing sensor analyte data are described in more detail herein and in U.S. Pat. Nos. 7,310,544 and 6,931,327 and U.S. Patent Publication Nos. 2005/0043598, 2007/0032706, 2007/0016381, 2008/0033254, 2005/0203360, 2005/0154271, 2005/0192557, 2006/0222566, 2007/0203966 and 2007/0208245, all of which are incorporated herein by reference in their entireties.

Analyte sensor 10 is configured to measure a concentration or level of the analyte in the host. The term analyte is further defined by U.S. App. No. 2019/0336053. In some embodiments, analyte sensor 10 is a subcutaneous, transdermal (e.g., transcutaneous), or intravascular device. Analyte sensor 10 can use any method of analyte-measurement, including enzymatic, chemical, physical, electrochemical, spectrophotometric, polarimetric, calorimetric, iontophoretic, radiometric, immunochemical, and the like. Additional details relating to a continuous glucose sensor are provided in paragraphs [0072]-[0076] of U.S. application Ser. No. 13/827,577. Paragraphs [0072]-[0076] of U.S. application Ser. No. 13/827,577 are incorporated herein by reference. In certain embodiments, analyte sensor 10 is a glucose sensor. However, any other analyte sensor, such as a potassium sensor, a lactate sensor, an ammonia sensor, a creatinine sensor, or the like, are all within the scope of the disclosure. In some embodiments, analyte sensor 10 may be a multi-analyte sensor configured to sense multiple analytes (e.g., glucose, potassium, lactate, and/or others).

With further reference to FIG. 1A, display devices 110, 120, 130, and/or 140 can be configured for displaying (and/or alarming) displayable sensor information that may be transmitted by sensor electronics circuitry 12 (e.g., in a customized data package that is transmitted to the display devices based on their respective preferences). Each of display devices 110, 120, 130, or 140 may respectively include a display such as touchscreen display 112, 122, 132, and/or 142 for displaying sensor information and/or analyte data to a user and/or receiving inputs from the user. For example, a graphical user interface (GUI) may be presented to the user for such purposes. In certain embodiments, the display devices may include other types of user interfaces, such as a voice user interface instead of or in addition to a touchscreen display for communicating sensor information to the user of the display device and/or receiving user inputs. In certain embodiments, one, some, or all of display devices 110, 120, 130, 140 may be configured to display or otherwise communicate the sensor information as it is communicated from sensor electronics circuitry 12 (e.g., in a data package that is transmitted to respective display devices), without any additional prospective processing required for calibration and/or real-time display of the sensor data.

The plurality of display devices 110, 120, 130, 140 depicted in FIG. 1A may include a custom or proprietary display device, for example, analyte display device, especially designed for displaying certain types of displayable sensor information associated with analyte data received from sensor electronics circuitry 12 (e.g., a numerical value and/or an arrow, in certain embodiments). In certain embodiments, one of the plurality of display devices 110, 120, 130, 140 includes a smartphone, such as display device 150, based on an Android, IOS, or another operating system configured to display a graphical representation of the continuous sensor data (e.g., including current and/or historic data). In certain embodiments, health management system 100 further includes a medical delivery device (e.g., an insulin pump or pen). Sensor electronics circuitry 12 may be configured to transmit sensor information and/or analyte data to medical delivery device. The medical delivery device (not shown) may be configured to administer a certain dosage of insulin or another medicament to the user based on the sensor information and/or analyte data (e.g., which may include a recommended insulin dosage) received from the sensor electronics circuitry 12.

FIG. 1B illustrates a more detailed view of health management system 100 including a display device 150 that is communicatively coupled to SS 8. In certain embodiments, display device 150 may be any one of display devices 110, 120, 130, and 140 of FIG. 1A. The communication path between SS 8 and display device 150 is shown as wireless communication path 180. In certain embodiments, SS 8 and display device 150 are configured to wirelessly communicate over wireless communication path 180 using short range and/or distance wireless communication protocols.

Examples of short range and/or distance wireless communication protocols include Bluetooth and Bluetooth Low Energy (BLE) protocols. In certain embodiments, other short range wireless communications may include Near Field Communications (NFC), radio frequency identification (RFID) communications, IR (infra-red) communications, optical communications. In certain embodiments, wireless communication protocols other than short range and/or distance wireless communication protocols may be used for wireless communication path 180, such as WiFi Direct. Display device 150 and/or SS 8 may also be configured to connect to network 190 (e.g., local area network (LAN), wide area network (WAN), the Internet, etc.). For example, display device 150 may connect to network 190 via a wired (e.g., Ethernet) or wireless (e.g., WLAN, wireless WAN, cellular, Mesh network, personal area network (PAN) etc.) interface.

Health management system 100 may additionally include server system 134, which in turn includes server 135 that is coupled to storage 136 (e.g., one or more computer storage systems, cloud-based storage systems and/or services, etc.). In certain embodiments, server system 134 may be located or execute in a public or private cloud. In certain embodiments, server system 134 is located or executes on-premises (“on-prem”). As discussed, server system 134 is configured to receive, collect, and/or monitor information, including analyte data and related information, as well as encryption/authentication information from SS 8 and/or display device 150. Such information may include input responsive to the analyte data or input (e.g., the user's glucose measurements and other physiological/behavioral information) received in connection with an analyte monitoring or sensor application running on SS 8 or display device 150. This information may be stored in storage 136 and may be processed, such as by an analytics engine capable of performing analytics on the information. An example of an analyte sensor application that may be executable on display device 150 is analyte sensor application 121, as further described below.

Display device 150 and SS 8 are able to communicate with server system 134 through network 190. The communication path between display device 150 and server system 134 is shown as communication path 181 via network 190. The communication path between SS 8 and server system 134 is shown as communication path 182 via network 190.

FIG. 1B also illustrates the components of SS 8 in further detail. As shown, in certain embodiments, SS 8 includes analyte sensor 10 coupled to sensor electronics circuitry 12. Sensor electronics circuitry 12 includes sensor measurement circuitry (SMC) 13 that is coupled to analyte sensor 10 for processing and managing sensor data. SMC 13 may also be coupled to processor 11. In some embodiments, processor 11 may perform part or all of the functions of the SMC 13 for obtaining and processing sensor measurement values from analyte sensor 10. Processor 11 may also be coupled to storage 14 and real time clock (RTC) 17 for storing and tracking sensor data. Processor 11 may further be coupled to one or more input sensor(s) 21, such as a force sensor 22 and an accelerometer 23, for detecting user input (e.g., user presses and/or taps on the SS 8).

In some embodiments, the obtaining and processing of sensor measurement values and/or user input may be managed by an analyte sensor application 18 stored in storage 14. For example, as shown, storage 14 stores analyte sensor application 18 that, when executed using processor 11, causes the processor 11 to receive and process sensor measurement values from analyte sensor 10. In addition, analyte sensor application 18, when executed using processor 11, may cause the processor 11 to receive and process user input (e.g., touch input) from input sensor(s) 21 in order to identify user presses, taps, and patterns or presses and/or taps on the SS 8. In some embodiments, analyte sensor application 18 is implemented as firmware that is executed by processor 11 to provide control of hardware elements (e.g., input sensor(s) 21, connectivity interface 15, RTC 17, SMC 13, etc.) included in SS 8.

In addition, processor 11 may be further coupled to a connectivity interface 15, which includes a radio unit or transceiver (TRX) 16 for sending sensor data and receiving requests and commands from an external device, such as display device 150. Connectivity interface 15 may further include a RF wakeup circuit 19 for causing the connectivity interface 15 and/or sensor electronics circuitry 12 to wake from a low-power state in response to RF signals (e.g., an on/off keying (OOK) pattern) received from an external device, such as display device 150. As used herein, the term transceiver generally refers to a device or a collection of devices that enable SS 8 to (e.g., wirelessly) transmit and receive data. SS 8 may further include storage 14 and RTC 17 for storing and tracking sensor data. It is contemplated that, in some embodiments, the SMC 13 may carry out all the functions of the processor 11 and vice versa.

Transceiver 16 may be configured with the necessary hardware and wireless communications protocols for enabling wireless communications between SS 8 and other devices, such as display device 150 and/or server system 134. For example, as described above, transceiver 16 may be configured with the necessary hardware and communication protocols to establish a Bluetooth or BLE connection with display device 150. As one of ordinary skill in the art appreciates, in such an example, the necessary hardware may include a Bluetooth or BLE security manager and/or other Bluetooth or BLE related hardware/software modules configured for Bluetooth or BLE communications standards. In some embodiments where SS 8 is configured to establish an independent communication path with server system 134, transceiver 16 may be configured with the necessary hardware and communication protocols (e.g., long range wireless cellular communication protocol, such as, GSM, CDMA, LTE, VOLTE, 3G, 4G, and 5G communication protocols, WiFi communication protocols, such as 802.11 communication protocols, etc.) for establishing a wireless connection to network 190 to connect with server system 134. As discussed elsewhere, other short range protocols, may also be used for communication between display device 150 and a SS 8 such as NFC, RFID, etc.

FIG. 1B similarly illustrates the components of display device 150 in further detail. As shown, display device 150 includes connectivity interface 128, processor 126, memory 127, one or more sensor(s) 163, a display 125 for presenting a graphical user interface (GUI), and a storage 123. A bus (not shown here) may be used to interconnect the various elements of display device 150 and transfer data between these elements. Connectivity interface 128 includes a transceiver (TRX) 129 used for receiving sensor data from SS 8 and for sending requests, instructions, and/or data to SS 8 as well as server system 134. Transceiver 129 is coupled to other elements of display device 150 via connectivity interface 128 and/or the bus. Transceiver 129 may include multiple transceiver modules operable on different wireless standards. For example, transceiver 129 may be configured with one or more communication protocols, such as wireless communication protocol(s) for establishing a wireless communication path with network 190 and/or short range wireless communication protocol(s) (e.g., Bluetooth or BLE) for establishing a wireless communication path 180 with SS 8.

Additionally, connectivity interface 128 may in some cases include additional components for controlling radio and/or wired connections, such as baseband and/or Ethernet modems, audio/video codecs, and so on. Sensor(s) 163 may include, but is not limited to, accelerometer(s), gyroscope(s), global positioning system (GPS) sensor(s), heart rate sensor(s), etc. Note that while sensor(s) 163 are shown integral to the display device 150, in certain embodiments, one or more of sensor(s) 163 be standalone sensors (e.g., separate from the display device 150).

In some embodiments, when a standardized communication protocol is used between display device 150 and SS 8, commercially available transceiver circuits may be utilized that incorporate processing circuitry to handle low level data communication functions such as the management of data encoding, transmission frequencies, handshake protocols, security, and the like. In such embodiments, processor 126 of display device 150 and/or processor 11 of SS 8 may not need to manage these activities, but instead provide desired data values for transmission, and manage high level functions such as power up or down, set a rate at which messages are transmitted, and the like. Instructions and data values for performing these high level functions can be provided to the transceiver circuits via a data bus and transfer protocol established by the manufacturer of transceivers 129 and 16. However, in embodiments where a standardized communication protocol is not used between transceivers 129 and 16 (e.g., when non-standardized or modified protocols are used), processors 126 and 11 may be configured to execute instructions associated with proprietary communications protocols (e.g., one or more of the communications protocols described herein) to control and manage their respective transceivers. In addition, when non-standardized or modified protocols are used, customized circuitries may be used to service such protocols.

Processor 126 may include processor sub-modules, including, by way of example, an applications processor that interfaces with and/or controls other elements of display device 150 (e.g., connectivity interface 128, analyte sensor application 121 (hereinafter “sensor application 121”), co-located application(s) 124, display 125, sensor(s) 163, memory 127, storage 123, etc.). In certain embodiments, processor 126 is configured to perform functions related to device management, such as, managing lists of available or previously paired devices, information related to network conditions (e.g., link quality and the like), information related to the timing, type, and/or structure of messaging exchanged between SS 8 and display device 150, and so on.

Processor 126 may include and/or be coupled to circuitry, such as logic circuits, memory, a battery and power circuitry, and other circuitry drivers for periphery components and audio components. Processor 126 and any sub-processors thereof may include logic circuits for receiving, processing, and/or storing data received and/or input to display device 150. Processor 126 and any sub-processors thereof may also include logic circuits for receiving, processing, and/or storing data to be transmitted or delivered by display device 150. As described above, processor 126 may be coupled by a bus to display 125, connectivity interface 128, storage 123, etc. Hence, processor 126 may receive and process electrical signals generated by these respective elements and thus perform various functions. By way of example, processor 126 may access stored content from storage 123 and memory 127 at the direction of analyte sensor application 121, and process the stored content to be displayed by display 125. Additionally, processor 126 may process the stored content for transmission via connectivity interface 128 to SS 8 and/or server system 134. Display device 150 may include other peripheral components not shown in detail in FIG. 1B.

In certain embodiments, memory 127 may include volatile memory, such as random access memory (RAM) for storing data and/or instructions for software programs and applications, such as analyte sensor application 121 and co-located application(s) 124. Display 125 presents a GUI associated with operating system 162 and/or analyte sensor application 121. In various embodiments, a user may interact with analyte sensor application 121 via a corresponding GUI presented on display 125. By way of example, display 125 may be a touchscreen display that accepts touch input. Analyte sensor application 121 may process and/or present analyte-related data received by display device 150 and present such data via display 125. Additionally, analyte sensor application 121 may be used to obtain, access, display, control, and/or interface with analyte data and related messaging and processes associated with SS 8 (e.g., and/or any other medical device (e.g., insulin pump or pen) that are communicatively coupled with display device 150), as is described in further detail herein.

Storage 123 may be a non-volatile storage for storing software programs, instructions, data, etc. For example, storage 123 may store analyte sensor application 121 that, when executed using processor 126, for example, receives input (e.g., by a conventional hard/soft key or a touch screen, voice detection, or other input mechanism), and allows a user to interact with the analyte data and related content via display 125. Similarly, storage 123 may store co-located application(s) 124 that, when executed using processor 126, for example, receives input (e.g., by a conventional hard/soft key or a touch screen, voice detection, or other input mechanism), and allows a user to interact with the other non-analyte related data and related content via display 125.

In various embodiments, storage 123 may also store user input data and/or other data collected by display device 150 (e.g., input from other users gathered via analyte sensor application 121). Storage 123 may further be used to store volumes of analyte data received from SS 8 (or any other medical data received from other medical devices (e.g., insulin pump, pen, etc.) for later retrieval and use, e.g., for determining trends and triggering alerts.

As described above, SS 8, in certain embodiments, gathers analyte data from analyte sensor 10 and transmits the same or a modified version of the collected data to display device 150. Data points regarding analyte values may be gathered and transmitted over the life of analyte sensor 10 (e.g., in the range of 1 to 30 days or more). New measurements may be transmitted often enough to adequately monitor analyte levels. As described above, in certain embodiments, rather than having the transmission and receiving circuitry of each of SS 8 and display device 150 continuously communicate, SS 8 and display device 150 may regularly and/or periodically establish a communication channel among each other.

Thus, in such embodiments, SS 8 may, for example, communicate with display device 150 at predetermined time intervals. The duration of the predetermined time interval can be selected to be long enough so that SS 8 does not consume too much power by transmitting data more frequently than needed, yet frequent enough to provide substantially real-time sensor information (e.g., measured glucose values or analyte data) to display device 150 for output (e.g., via display 125) to the user. While the predetermined time interval is every five minutes in some embodiments, it is appreciated that this time interval can be varied to be any desired length of time. In other embodiments, transceivers 129 and 16 may be continuously communicating. For example, in certain embodiments, transceivers 129 and 16 may establish a session or connection there between and continue to communicate together until the connection is lost.

Analyte sensor application 121 may be downloaded, installed, and initially configured/setup on display device 150. For example, display device 150 may obtain analyte sensor application 121 from server system 134, or from another source, such as an application store or the like, via a network, e.g., network 190. Following installation and setup, analyte sensor application 121 may be configured to access, process, and/or interface with analyte data (e.g., whether stored on server system 134, locally from storage 123, from SS 8, or any other medical device). By way of example, analyte sensor application 121 may present a menu that includes various controls or commands that may be executed in connection with the operation of SS 8, display device 150, one or more other display devices (e.g., display device 110, 130, 140, etc.), and/or one or more other partner devices, such as an insulin pump. For example, analyte sensor application 121 may be used to interface with or control other display and/or partner devices, for example, to deliver or make available thereto analyte data, including, for example, by receiving/sending analyte data directly to the other display and/or partner device and/or by sending an instruction for SS 8 and the other display and/or partner device to be connected.

After downloading analyte sensor application 121, as one of the initial steps, the user may be directed by analyte sensor application 121 to wirelessly connect display device 150 to the user's SS 8, which the user may have already placed on their body. A wireless communication path 180 between display device 150 and SS 8 allows SS 8 to transmit analyte measurements to display device 150 and for the two devices to engage in any of the other interactions described herein.

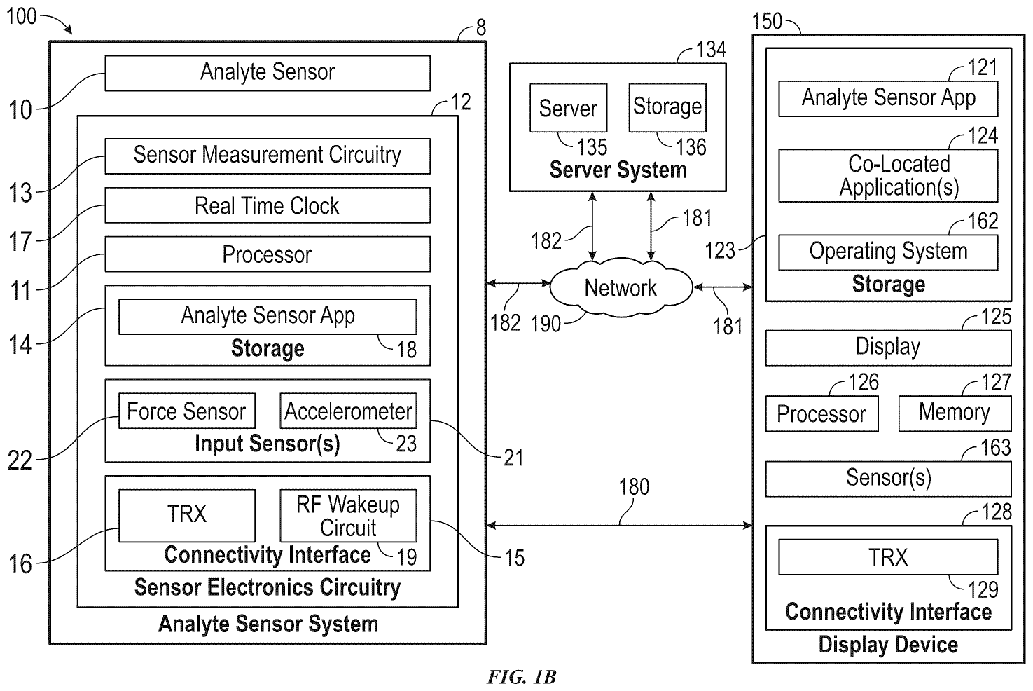

FIG. 2A illustrates a perspective view of an outer housing 210 of SS 8, according to certain embodiments disclosed herein. In some embodiments, the outer housing 210 may include a clamshell design. SS 8 may further include, for example, a power source (e.g., a battery 24) for providing power to analyte sensor 10 and analyte sensor electronics circuitry 12.

As shown in FIG. 2A, the outer housing 210 may include aperture 230 disposed through a portion of outer housing 210 and adapted for analyte sensor 10 and needle insertion through a bottom of SS 8. In some embodiments, aperture 230 may include a channel or an elongated slot. SS 8 may further include an adhesive patch 220 configured to secure SS 8 to skin of the host. The adhesive patch 220 may include an adhesive suitable for skin adhesion, for example, a pressure sensitive adhesive (e.g., acrylic, rubber-based, or other suitable type) bonded to a carrier substrate (e.g., spun lace polyester, polyurethane film, or other suitable type) for skin attachment, although any suitable type of adhesive may be implemented.

FIG. 2B illustrates a bottom perspective view of the outer housing 210 of the SS 8 of FIG. 2A. As shown, adhesive patch 220 may feature an aperture aligned with aperture 230 such that sensor 10 may pass through a bottom of SS 8 and through adhesive patch 220.

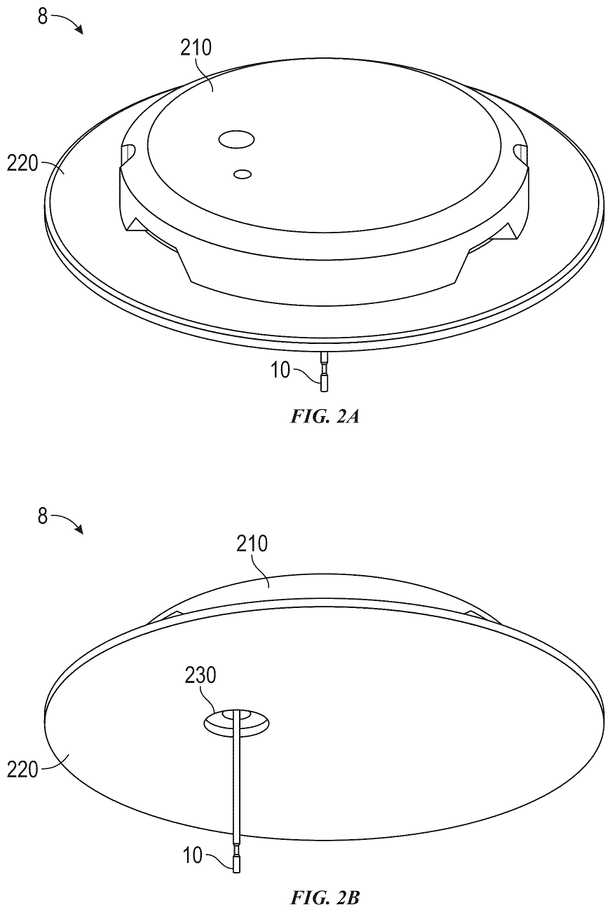

FIG. 3 illustrates a cross-sectional view of the outer housing 210 of SS 8 of FIG. 2A and FIG. 2B. As shown, SS 8 includes analyte sensor 10, sensor electronics circuitry 12, and a power source, which may include battery 24.

In some embodiments, one or more input sensors 21 may be coupled to or otherwise in communication with outer housing 210 of SS 8. For example, a force sensor 22 may be coupled to an interior surface of the outer housing 210 and/or otherwise disposed within the outer housing 210 and configured to detect touch input and/or other forces applied to the outer housing 210 (e.g., a user tapping and/or pressing on the outer housing 210). In another example, an accelerometer 23 may be coupled to an interior surface of the outer housing 210 and/or otherwise disposed within the outer housing 210 and configured to detect an acceleration applied to the outer housing 210 (e.g., a user tapping on the outer housing 210, movement of the outer housing 210, etc.) and/or an orientation of the outer housing 210. Any other type of sensor capable of detecting user input (e.g., user taps, user presses, forces, accelerations, etc.) and/or orientations may be implemented in various embodiments.

### On-Demand Transmission of Analyte Data

As described above, in certain embodiments described herein, sensor electronics circuitry 12 is configured to transmit analyte data to the display device 150 and perform other functions on-demand, instead of waiting for a fixed period of time before performing such functions. For example, in certain embodiments described herein, a user may trigger on-demand transmission of analyte data via touch input, such as by pressing and/or tapping on the outer housing 210 of the SS 8. In some embodiments, each unique pattern of presses and/or taps may trigger on-demand transmission of a different type of analyte data.

In some embodiments, the sensor electronics circuitry 12 may implement a two-phase wakeup technique to distinguish between intentional user input requesting analyte data to be transmitted, and inadvertent input that causes the sensor electronics circuitry 12 to frequently wake from the low-power state, which may decrease battery life. In addition, on-demand transmission of analyte data may be triggered via the display device 150 (e.g., via a button on a touchscreen interface) by causing the display device 150 to generate a radiofrequency (RF) signal pattern, such as an on/off keying (OOK) pattern, that causes the connectivity interface 15 in the sensor electronics circuitry 12 to wake from the low-power state. Accordingly, a user is able to obtain updated analyte data on-demand, without significantly impacting the battery life of the SS 8.

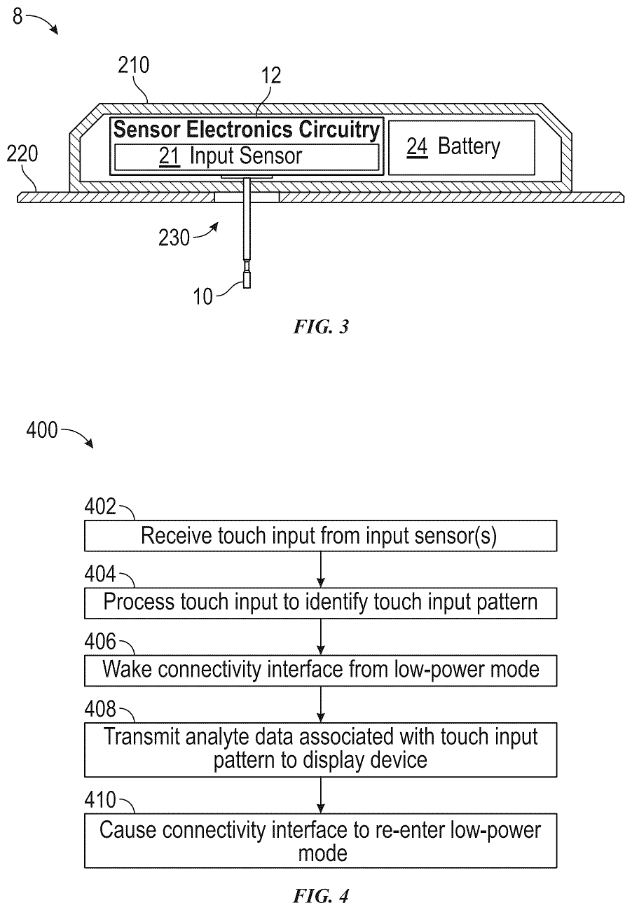

Although the techniques of FIGS. 4-8 as described individually for clarity of explanation, in various embodiments, any of the techniques may be combined with one or more other techniques in order to transition the sensor electronics circuitry 12 into and out of the low-power state and/or to facilitate the on-demand transmission of analyte data to the display device 150.

### Example Operations for On-Demand Transmission of Analyte Data Based on Touch Input

FIG. 4 is a flow diagram illustrating example operations for triggering on-demand transmission of analyte data based on touch input, according to certain embodiments disclosed herein. The operations 400 may be performed by one or more components of an analyte sensor system, such as the sensor electronics circuitry 12 of the SS 8. For example, in some embodiments, one or more components of the sensor electronics circuitry 12 may be configured to perform operations 400, such as the processor 11, the analyte sensor app 18, the input sensor(s) 21, and/or the transceiver (TRX) 16 of the connectivity interface 15 illustrated in FIG. 1B.

Operations 400 begin in step 402, with the sensor electronics circuitry 12 (e.g., the analyte sensor app 18 being executed by processor 11) receiving touch input from the input sensor(s) 21. For example, force sensor 22 and/or accelerometer 23, which may be on or in communication with outer housing 210 of the SS 8, may receive touch input while the device is being worn by the user and transmit one or more signals corresponding to the touch input to the analyte sensor app 18 being executed by processor 11. As defined herein, touch input may include one or more user taps (e.g., a single-tap, a double-tap, a triple-tap, . . . n consecutive taps), one or more user presses (e.g., a short-press, a long-press of a specified duration, such as 1 second, 2 seconds, . . . n seconds), or any combination thereof on the SS 8, such as on the outer housing 210 of the SS 8. In embodiments in which two or more input sensors 21 (e.g., two or more force sensors 22) are implemented, touch input may include directional touch input (e.g., a swipe from the left, right, top, or bottom of a surface of the SS 8), multi-touch input (e.g., gestures performed by two or more fingers on the SS 8), a pinch or spread gesture (e.g., pinching two or more fingers together or spreading two or more fingers apart on a surface of the SS 8), or any combination thereof.

In step 404, the analyte sensor app 18 processes the touch input to identify one or more touch input patterns. For example, the analyte sensor app 18 may receive signals from force sensor 22 that correspond to (i) a long-press on the outer housing 210 of the SS 8, (ii) a short-press followed by a long-press on the outer housing 210 of the SS 8, (iii) a series of short-presses on the outer housing 210 of the SS 8, (iv) a series of long-presses on the outer housing 210 of the SS 8, or any combination thereof. In another example, the analyte sensor app 18 may receive signals from accelerometer 23 that correspond to a tap or a pattern of taps on the outer housing 210 of the SS 8. In yet another example, the analyte sensor app 18 may receive signals from both force sensor 22 and accelerometer 23 that correspond to a combination of user presses and user taps, such as (i) a single-tap followed by a long-press, (ii) a double-tap followed by a long-press, (iii) n-taps followed by a long press, (iv) a long-press followed by one or more taps, (v) a short-press followed by one or more taps, or any combination thereof. In such embodiments, by implementing both force sensor 22 and accelerometer 23 to detect touch input, the incidence of false detection (e.g., due to inadvertent user input) of a touch input pattern may be reduced, extending the battery life of the SS 8.

As described above, the sensor electronics circuitry 12 may initially connect and authenticate with the display device 150. Once the sensor electronics circuitry 12 and the display device 150 have paired and bonded, each of the devices will store information about the other device, and pairing and bonding will not be necessary during reconnections. Subsequently, in order to conserve power, the connectivity interface 15 (and/or one or more other components of the sensor electronics circuitry 12) remains in a low-power state and wakes from the low-power state to reconnect to the display device 150 only periodically, such as every 5 minutes, in order to transmit updated analyte data.

In various embodiments, while the connectivity interface 15 is in this low-power state, in step 406, the analyte sensor app 18 causes the connectivity interface 15 to wake from the low-power state (e.g., by transitioning to the operational state) upon identifying a touch input pattern based on touch input received from the input sensor(s) 21. In step 408, the analyte sensor app 18 then transmits analyte data (e.g., analyte data that was acquired while the connectivity interface 15 was in the low-power state) to the display device 150 via the connectivity interface 15. After receiving the analyte data, the display device 150 may display information corresponding to the analyte data via analyte sensor app 121.

In step 410, the connectivity interface 15 re-enters the low-power state. For example, the connectivity interface 15 may be programmed or otherwise configured to re-enter the low-power state after the analyte data is transmitted to the display device 150 and/or the analyte sensor app 18 may send a signal to the connectivity interface 15 to cause the connectivity interface 15 to re-enter the low-power state after analyte data is transmitted to the display device 150. The connectivity interface 15 may then resume the normal, periodic cycle of waking from the low-power state to transmit updated analyte data. In addition, the sensor electronics circuitry 12 may continue to acquire analyte data via analyte sensor 10 while the connectivity interface 15 is in the low-power state.

In some embodiments, in step 408, the type of analyte data that is transmitted by the analyte sensor app 18 to the display device 150 is based on the touch input pattern identified by the analyte sensor app 18 in step 404. For example, a first type of analyte data (e.g., estimated glucose values) may be transmitted to the display device 150 based on a first touch input pattern (e.g., a single-tap or a single-tap followed by a long-press) being detected in step 404, a second type of analyte data (e.g., estimated ketone values or lactate values) may be transmitted to the display device 150 based on a second touch input pattern (e.g., a double-tap or a double-tap followed by a long-press) being detected in step 404, and/or a third type of analyte data (e.g., estimated O₂ values) may be transmitted to the display device 150 based on a third touch input pattern (e.g., a triple-tap or a triple-tap followed by a long-press) being detected in step 404.

In general, any number of different touch input patterns, corresponding to any number of different types of analyte data, may be identified by the analyte sensor app 18 in order to trigger the on-demand transmission of analyte data to the display device 150. In this manner, a user can quickly and easily cause a specific type of analyte data in which they are interested to be transmitted to the display device 150 by inputting a corresponding touch input pattern on the SS 8, without needing to wait for a fixed period of time to expire. Additionally or alternatively to causing analyte data to be transmitted to the display device 150, in various embodiments, different types of touch input (e.g., a double-tap, a triple-tap, a long-press, etc.) could cause the sensor electronics circuitry 12 to perform different functions, such as a sensor health test or a device self-test.

### Example Operations for Detecting a Combination of Touch Input for False Wake Rejection

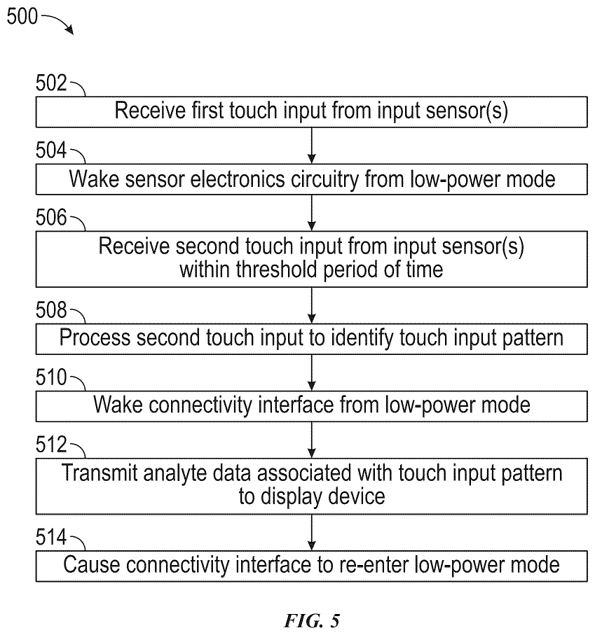

FIG. 5 is a flow diagram illustrating example operations for detecting a combination of touch input for false wake rejection, according to certain embodiments disclosed herein. The operations 500 may be performed by one or more components of an analyte sensor system, such as the sensor electronics circuitry 12 of the SS 8. For example, in some embodiments, one or more components of the SS 8 may be configured to perform operations 500, such as the processor 11, the analyte sensor app 18, the input sensor(s) 21, and/or the TRX 16 of the connectivity interface 15 illustrated in FIG. 1B.

Operations 500 begin in step 502, with the sensor electronics circuitry 12 receiving first touch input from the input sensor(s) 21. For example, the sensor electronics circuitry 12 may receive a single-tap, a double-tap, or a long-press via the input sensor(s) 21. In general, however, any of the configurations of input sensor(s) 21 and types of touch input described herein (e.g., described above in conjunction with FIG. 4) may be implemented in the operations of FIG. 5.

In step 504, in response to the first touch input, the sensor electronics circuitry 12 (e.g., processor 11) wakes from a low-power state. In some embodiments, any signal(s) received from the input sensor(s) 21 cause the sensor electronics circuitry 12 to wake from the low-power state. In other embodiments, only certain signals (corresponding to certain types of touch input) received from the input sensor(s) 21 will cause the sensor electronics circuitry 12 to wake from the low-power state.

In step 506, the sensor electronics circuitry 12 (e.g., the analyte sensor app 18 being executed by processor 11) receives second touch input from the input sensor(s) 21 within a threshold period of time (e.g., 0.5 seconds, 1 second, 2 seconds, etc.) of receiving the first touch input from the input sensor(s) 21. For example, in steps 502 and 506, the sensor electronics circuitry 12 may receive a single-tap as first touch input followed by a long-press, within the threshold period time of the first touch input, as second touch input.

In step 508, the analyte sensor app 18 processes the second touch input to identify a touch input pattern. In step 510, the analyte sensor app 18 causes the connectivity interface 15 to wake from the low-power state upon identifying a touch input pattern in the second touch input received from the input sensor(s) 21.

In step 512, the analyte sensor app 18 transmits analyte data (e.g., analyte data that was acquired while the connectivity interface 15 was in the low-power state) to the display device 150 via the connectivity interface 15. In step 514, the connectivity interface 15 re-enters the low-power state.

In various embodiments, if second touch input is not received in step 506 within the threshold period of time of the first touch input and/or if a touch input pattern is not identified in step 508, then the sensor electronics circuitry 12 re-enters the low-power state.

As described above, the technique of FIG. 5 may be combined with any of the other techniques described herein (e.g., the techniques of FIGS. 4 and/or 6-8). For example, in step 508, the analyte sensor app 18 may identify a touch input pattern that corresponds to a specific type of analyte data and, in step 512, cause that specific type of analyte data to be transmitted to the display device 150.

### Example Operations for Timeout Period after Wakeup

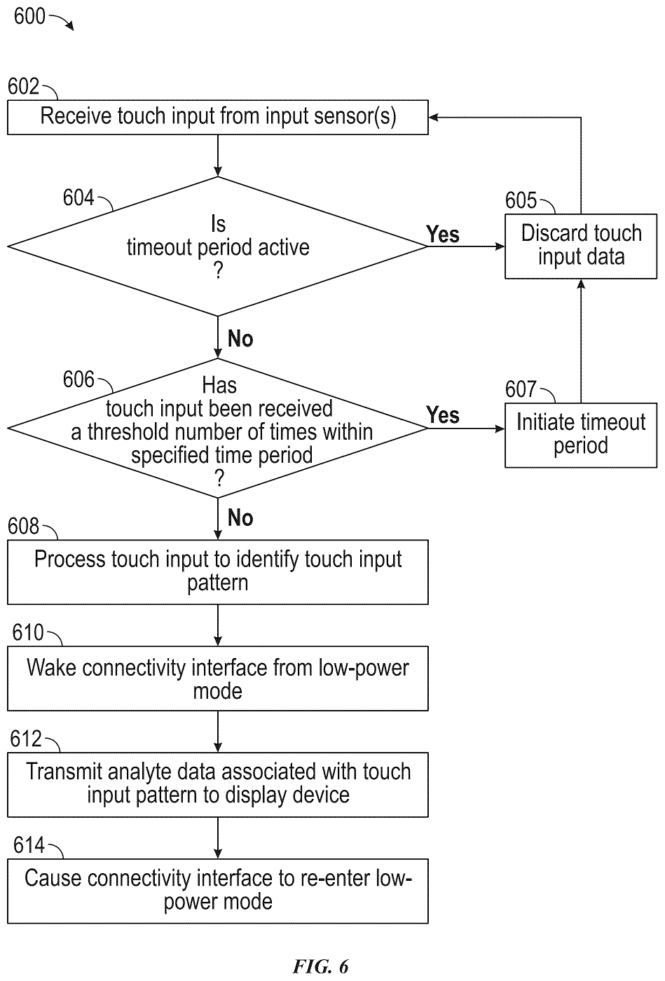

FIG. 6 is a flow diagram illustrating example operations for implementing a timeout period after waking the sensor electronics circuitry 12 and/or connectivity interface 15 from a low-power state, according to certain embodiments disclosed herein. The operations 600 may be performed by one or more components of an analyte sensor system, such as the sensor electronics circuitry 12 of the SS 8. For example, in some embodiments, one or more components of the SS 8 may be configured to perform operations 600, such as the processor 11, the analyte sensor app 18, the input sensor(s) 21, and/or the TRX 16 of the connectivity interface 15 illustrated in FIG. 1B.

Operations 600 begin in step 602, with the sensor electronics circuitry 12 (e.g., the analyte sensor app 18 being executed by processor 11) receiving touch input from the input sensor(s) 21. For example, the sensor electronics circuitry 12 may receive any type of touch input described herein from any configuration of input sensor(s) 21 described herein.

In step 604, the sensor electronics circuitry 12 determines whether a timeout period is active. If a timeout period is active, in step 605, the sensor electronics circuitry 12 discards the touch input data.

If a timeout period is not active, in step 606, the sensor electronics circuitry 12 determines whether touch input has been received a threshold number of times within a predetermined period of time. For example, in step 606, the sensor electronics circuitry 12 may determine whether touch input has caused the sensor electronics circuitry 12 and/or connectivity interface 15 to wake from a low-power state a threshold number of times (e.g., 2, 3, 4, etc.) within a predetermined period of time (e.g., 30 seconds, 1 minute, 2 minutes, etc.). Such repeated wakeups may be caused by inadvertent user input and may cause inadvertent transmission of analyte data, reducing the battery life of the SS 8. Accordingly, if touch input has been received a threshold number of times within a predetermined period of time (e.g., causing the sensor electronics circuitry 12 and/or connectivity interface 15 to wake from a low-power state and transmit analyte data), then, in step 607, the sensor electronics circuitry 12 initiates a timeout period (e.g., 1 minute, 2 minutes, 3 minutes, etc.).

If touch input has not been received a threshold number of times within the predetermined period of time, then, in step 608, the sensor electronics circuitry 12 processes the touch input to identify a touch input pattern.

In step 610, the sensor electronics circuitry 12 causes the connectivity interface 15 to wake from the low-power state. In step 612, the analyte sensor app 18 transmits analyte data to the display device 150 via the connectivity interface 15. Then, in step 614, the connectivity interface 15 re-enters the low-power state.

As described above, the technique of FIG. 6 may be combined with any of the other techniques described herein (e.g., the techniques of FIGS. 4, 5, 7, and/or 8). For example, in step 608, the analyte sensor app 18 may identify a touch input pattern that corresponds to a specific type of analyte data and, in step 612, cause that specific type of analyte data to be transmitted to the display device 150.

### Example Operations for Orientation-Based False Wake Rejection

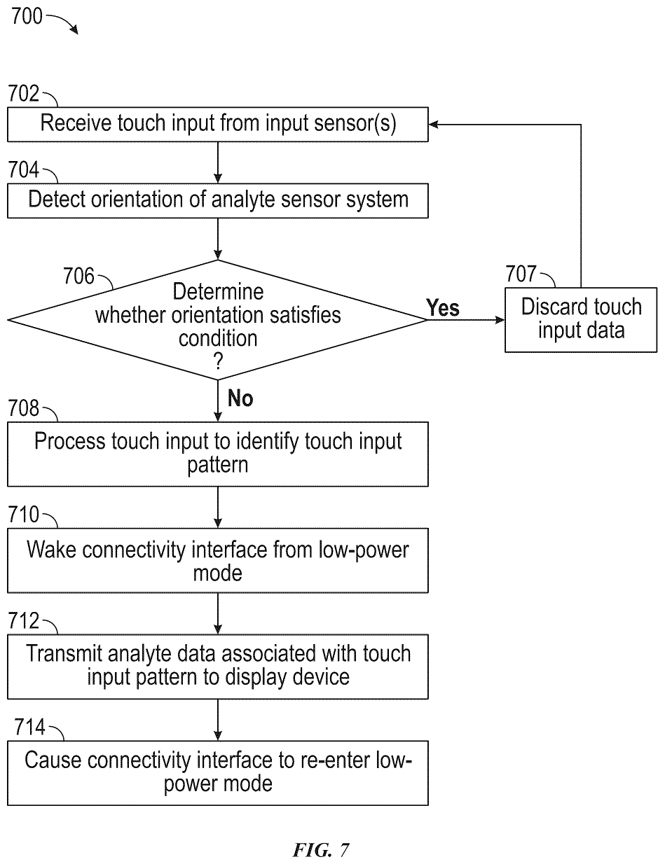

FIG. 7 is a flow diagram illustrating example operations for rejecting false wakeup of the SS 8 based on orientation, according to certain embodiments disclosed herein. The operations 700 may be performed by one or more components of an analyte sensor system, such as the sensor electronics circuitry 12 of the SS 8. For example, in some embodiments, one or more components of the SS 8 may be configured to perform operations 700, such as the processor 11, the analyte sensor app 18, the input sensor(s) 21, and/or the TRX 16 of the connectivity interface 15 illustrated in FIG. 1B.

Operations 700 begin in step 702, with the sensor electronics circuitry 12 (e.g., the analyte sensor app 18 being executed by processor 11) receiving touch input from the input sensor(s) 21. For example, the sensor electronics circuitry 12 may receive any type of touch input described herein from any configuration of input sensor(s) 21 described herein.

In step 704, the sensor electronics circuitry 12 detects an orientation of the SS 8, for example, based on one or more signals received from the input sensor(s) 21 (e.g., accelerometer 23). In step 706, the sensor electronics circuitry 12 determines whether the orientation satisfies a condition. In various embodiments, the condition is satisfied when the orientation corresponds to a position of the SS 8 in which the user is likely lying or sitting on top of the SS 8, which may cause a force or other touch input to be inadvertently applied to the SS8 (e.g., to the outer housing 210 of the SS 8).

If, in step 706, the orientation satisfies the condition, then, in step 707, the touch input data is discarded. If, on the other hand, in step 706, the orientation does not satisfy the condition, then, in step 708, the sensor electronics circuitry 12 processes the touch input to identify a touch input pattern.

In step 710, the sensor electronics circuitry 12 causes the connectivity interface 15 to wake from the low-power state. In step 712, the analyte sensor app 18 transmits analyte data to the display device 150 via the connectivity interface 15. In step 714, the connectivity interface 15 re-enters the low-power state.

In some embodiments, after a user applies the SS 8 to their body, the sensor electronics circuitry 12 may receive user input indicating a position of the SS 8 on the body of the user (e.g., a location on an arm of the user, a location on the back of the user, etc.). In such embodiments, in step 706, the sensor electronics circuitry 12 may determine, based on the position of the SS 8, whether the orientation of the SS 8 indicates that the user is likely lying, sitting, or otherwise applying a force (e.g., compression) or other input to the SS 8.

For example, if a signal received from the input sensor(s) 21 indicates that the SS 8 is in an orientation that corresponds to the user lying on their right side, and the user input received by the sensor electronics circuitry 12 indicates that the SS 8 is positioned on the right arm of the user, then the sensor electronics circuitry 12 would determine that the orientation satisfies the condition. If, on the other hand, a signal received from the input sensor(s) 21 indicates that the user is standing and/or upright, indicating that touch input received by the input sensor(s) 21 is likely intentional, in step 706, the sensor electronics circuitry 12 would determine that the orientation does not satisfy the condition. Then, in step 708, the sensor electronics circuitry 12 could identify a touch input pattern and, in step 710, cause the connectivity interface 15 to exit the low-power state to transmit analyte data to the display device 150 (step 712).

As described above, the technique of FIG. 7 may be combined with any of the other techniques described herein (e.g., the techniques of FIGS. 4-6 and/or 8). For example, in step 708, the analyte sensor app 18 may identify a touch input pattern that corresponds to a specific type of analyte data and, in step 712, cause that specific type of analyte data to be transmitted to the display device 150.

### Example Operations for On-Demand Transmission of Analyte Data Based on Proximity RE

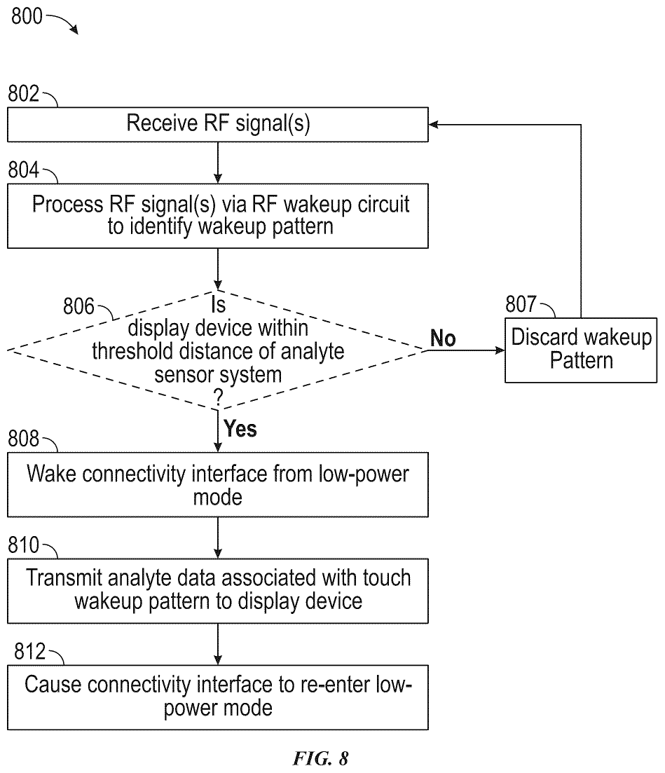

FIG. 8 is a flow diagram illustrating example operations for on-demand transmission of analyte data based on proximity RF, according to certain embodiments disclosed herein. The operations 800 may be performed by one or more components of an analyte sensor system and display device 150, such as the sensor electronics circuitry 12 of the SS 8. For example, in some embodiments, one or more components of the SS 8 and display device 150 may be configured to perform operations 800, such as the processor 11, the analyte sensor app 18, analyte sensor app 21, the input sensor(s) 21, the TRX 16 and RF wakeup circuit 19 of the connectivity interface 15, and/or the TRX 129 of the connectivity interface 128 illustrated in FIG. 1B.

Operations 800 begin in step 802, with the sensor electronics circuitry 12 (e.g., the analyte sensor app 18 being executed by processor 11) receiving RF signal(s) from the display device 150. In step 804, the RF wakeup circuit 19 processes the RF signal(s) to identify a wakeup pattern. In step 806, the RF wakeup circuit 19 and/or the sensor electronics circuitry 12 optionally determine whether the display device 150 is within a threshold distance of the SS 8 (e.g., based on a strength of the RF signal(s)).

If, in step 806, the RF wakeup circuit 19 and/or the sensor electronics circuitry 12 determines that the display device 150 is not within the threshold distance of the SS 8, then, in step 807, the wakeup pattern is discarded. If, on the other hand, in step 806, the RF wakeup circuit 19 and/or the sensor electronics circuitry 12 determines that the display device 150 is within the threshold distance (e.g., within 0.5 meters, 1 meter, 2 meters, etc.) of the SS 8, then, in step 808, the RF wakeup circuit 19 and/or the sensor electronics circuitry 12 causes the connectivity interface 15 to wake from the low-power state. In various embodiments, the RF wakeup circuit 19 processes incoming RF signals (e.g., to identify a wakeup pattern) while the connectivity interface 15 remains in a low-power state, providing additional power savings.

In step 810, the analyte sensor app 18 transmits analyte data to the display device 150 via the connectivity interface 15. Then, in step 812, the connectivity interface 15 re-enters the low-power state.

In various embodiments, the RF signal(s) transmitted by the display device 150 to the SS 8 may include an on/off keying (OOK) pattern. For example, TRX 129 (e.g., a BLE module) included in the display device 150 may generate an on/off pattern at a specific frequency, a specific set of frequencies, in a specific frequency range, or any combination thereof. In steps 802 and 804, the RF wakeup circuit 19 may listen for the OOK pattern while the sensor electronics circuitry 12 and/or connectivity interface 15 remain in a low-power state. Then, once the RF wakeup circuit 19 identifies the OOK pattern received from the display device 150, in step 808, the RF wakeup circuit 19 wakes the connectivity interface 15 (e.g., a BLE module) from the low-power state.

In some embodiments, a user may trigger on-demand analyte data to be transmitted from the SS 8 to the display device 150 by pressing a button on the display device 150, such as a virtual button included in analyte sensor app 121. In response to the user pressing the button, analyte sensor app 121 causes the TRX 129 (e.g., a BLE module) to generate the wakeup pattern (e.g., an OOK pattern). In some embodiments, the SS 8 may notifies the display device 150 (e.g., analyte sensor app 121) of the wakeup pattern that the RF wakeup circuit 19 is listening for during an initial pairing process between the SS 8 and display device 150.

Accordingly, analyte data can be requested from the SS 8 on-demand while conserving battery power. Additionally, implementing a threshold distance for waking the connectivity interface 15 and/or sensor electronics circuitry 12 may mitigate false wakeups and further conserve battery power.

In some embodiments, in step 810, the type of analyte data that is transmitted by the analyte sensor app 18 to the display device 150 is based on the wakeup pattern identified by the RF wakeup circuit 19 and/or analyte sensor app 18 in step 804. For example, a first type of analyte data (e.g., estimated glucose values) may be transmitted to the display device 150 based on a first wakeup pattern (e.g., a first OOK pattern) being detected in step 804, a second type of analyte data (e.g., estimated ketone values or lactate values) may be transmitted to the display device 150 based on a second wakeup pattern (e.g., a second OOK pattern) being detected in step 804, and/or a third type of analyte data (e.g., estimated O₂ values) may be transmitted to the display device 150 based on a third wakeup pattern (e.g., a third OOK pattern) being detected in step 804.

In general, any number of different wakeup patterns, corresponding to any number of different types of analyte data, may be identified by the RF wakeup circuit 19 and/or analyte sensor app 18 in order to trigger the on-demand transmission of analyte data to the display device 150. In this manner, a user can quickly and easily cause a specific type of analyte data in which they are interested to be transmitted to the display device 150 by touching a specific virtual button (e.g., corresponding to a specific type of analyte data being requested) on the display device 150 (e.g., via analyte sensor app 121), without needing to wait for a fixed period of time to expire.

Additionally or alternatively to causing analyte data to be transmitted to the display device 150, in various embodiments, different wakeup patterns could cause the sensor electronics circuitry 12 to perform different functions, such as a sensor health test or a device self-test. In some embodiments, a first wakeup pattern could be transmitted by the display device 150 to the RF wakeup circuit 19 to cause the sensor electronics circuitry 12 and/or connectivity interface 15 to wake from a low-power state, and then one or more additional wakeup patterns could be transmitted by the display device 150 to request one or more different types of analyte data to be sent to the display device 150 and/or one or more different functions to be performed by the SS 8.

While the SS 8 is in storage and/or prior to first use, the sensor electronics circuitry 12 may be placed into a low-power state to conserve battery power. Accordingly, in some embodiments, after a user applies the SS 8 to their body via an applicator, the RF wakeup circuit 19 may detect a wakeup pattern generated by the display device 150. In response to detecting the wakeup pattern, the sensor electronics circuitry 12 may initiate a wakeup routine that causes the sensor electronics circuitry 12 and/or connectivity interface 15 to exit a low-power state and optionally initiate a pairing sequence with a display device 150. Additionally, as described above, a user may press a virtual button on the display device 150 to generate the wakeup pattern and initiate the wakeup routine.

As described above, the technique of FIG. 8 may be combined with any of the other techniques described herein (e.g., the techniques of FIGS. 4-7).

### Example Operations for Low-Power Sensing Circuit

In various embodiments, the input sensor(s) 21 included in the sensor electronics circuitry 12 may implement a low-power state in which the input sensor(s) 21 periodically wake up for a short period of time (e.g., awake for 10 milliseconds every second, awake for 1-10% of each second, etc.) in order to check for touch input. If the input sensor(s) 21 detect user input, then the input sensor(s) 21 and/or sensor electronics circuitry 12 may enter a higher-sensitivity mode to further detect and confirm the touch input (e.g., by identifying a touch input pattern, as described above). For example, by causing the input sensor(s) 21 to wake for ˜10 ms every second, the sensor electronics circuitry 12 will be certain to detect a long-press that lasts one second or longer. The input sensor(s) 21 and/or sensor electronics circuitry 12 may then enter the higher-sensitivity mode to confirm that touch input (e.g., a long-press) is occurring.

Further, in embodiments that implement the Combination Input for False Wake Rejection technique described in conjunction with FIG. 5, the input sensor(s) 21 and/or sensor electronics circuitry 12 may further detect additional touch input (e.g., “second touch input”), such as a single-tap or double-tap that follows a long-press (e.g., “first touch input”) by the user. The analyte monitoring system may remain in the higher-sensitivity mode for a short period of time (e.g., 3-5 seconds) and re-enter the low-power state if no additional touch input is detected.

### Dynamically Adjusting BLE Transmission Power

In various embodiments, the wireless transmission power (e.g., BLE transmission power) of the connectivity interface 15 may be dynamically adjusted based on the input sensor(s) 21 and/or sensor electronics circuitry 12 detecting touch input. For example, BLE transmission power may be increased when touch input is detected in order to increase signal range of the connectivity interface 15 and connection reliability (e.g., between the SS 8 and the display device 150). In order to reduce power consumption and improve battery life, wireless transmission power may be decreased after analyte data has been transmitted from the SS 8 to the display device 150 and/or when no touch input has been detected via the input sensor(s) 21 for a threshold period of time.

### Wakeup During Application

In various embodiments, in order to apply the SS 8 to the body of a user, the user positions the SS 8 in an applicator. Then, when the user presses on the applicator to apply the SS 8 to their body, the force translates through the applicator and is detected via an input sensor 21 of the SS 8, such as a force sensor 22 that is in communication with the outer housing 210 of the SS 8. Alternatively, a user may press directly onto the SS 8 (e.g., directly onto the outer housing 210 of the SS 8) when applying the SS 8 to their body, such that the force is detected via an input sensor 21 (e.g., force sensor 22) positioned on or in communication with the outer surface 210 of the SS 8. In response to detecting the force applied to the SS 8 (e.g., via an applicator or via a user pressing directly on the SS 8), the sensor electronics circuitry 12 initiates a wakeup routine that causes one or more components in the sensor electronics circuitry 12 (e.g., the connectivity interface 15) to exit a low-power state and optionally initiate a pairing sequence with a display device 150.

### Example Clauses

Implementation examples are described in the following numbered clauses:

Clause 1: A system for on-demand transmission of analyte data, the system comprising: an analyte sensor; at least one input sensor; and analyte sensor electronics circuitry comprising a connectivity interface, the analyte sensor electronics circuitry configured to: process touch input data received via the at least one input sensor to identify a first touch input pattern; based on identifying the first touch input pattern, transition the connectivity interface from a low-power state to an operational state; upon transitioning the connectivity interface to the operational state, transmit first analyte data to a display device; and transition the connectivity interface from the operational state to the low-power state after transmitting the first analyte data to the display device.

Clause 2: The system of Clause 1, wherein the at least one input sensor comprises a force sensor, and the first touch input pattern comprises at least one long-press.

Clause 3: The system of any of Clauses 2 or 3, wherein the at least one input sensor comprises an accelerometer and a force sensor, and the first touch input pattern comprises: (i) at least one tap detected via the accelerometer, and (ii) at least one long-press detected via the force sensor.

Clause 4: The system of any of Clauses 1-3, wherein the analyte sensor electronics circuitry acquires the analyte data via the analyte sensor while the connectivity interface is in the low-power state.

Clause 5: The system of any of Clauses 1-4, wherein the analyte sensor electronics circuitry is further configured to: process additional touch input data received via the at least one input sensor to identify a second touch input pattern; based on identifying the second touch input pattern, transition the connectivity interface from the low-power state to the operational state; and upon transitioning the connectivity interface to the operational state, transmit second analyte data to the display device.

Clause 6: The system of Clause 5, wherein the first analyte data comprises a first analyte type that corresponds to the first touch input pattern, and the second analyte data comprises a second analyte type that corresponds to the second touch input pattern.

Clause 7: The system of any of Clauses 1-6, wherein the analyte sensor electronics circuitry is further configured to: receive additional touch input data via the at least one input sensor; and in response to determining that a timeout period is active, discard the additional touch input data.

Clause 8: The system of Clause 7, wherein the analyte sensor electronics circuitry is further configured to: determine that touch input has been received a threshold number of times within a predetermined period of time, or determine that the connectivity interface has been transitioned from the low-power state to the operational state the threshold number of times within the predetermined period of time; and initiate the timeout period.

Clause 9: The system of any of Clauses 1-8, wherein the analyte sensor electronics circuitry is further configured to, prior to processing the touch input data: transition a processor included in the analyte sensor electronics circuitry from a second low-power state to a second operational state in response to receiving initial touch input data via the at least one input sensor; and determine that the touch input data was received within a threshold period of time of the initial touch input data, wherein the analyte sensor electronics circuitry is configured to transition the connectivity interface from the low-power state to the operational state based at least in part on determining that the touch input data was received within the threshold period of time.

Clause 10: The system of any of Clauses 1-9, wherein the analyte sensor electronics circuitry is further configured to: detect a first orientation of the analyte sensor electronics circuitry via the at least one input sensor; and determine that the first orientation does not satisfy a condition, wherein the analyte sensor electronics circuitry is configured to transition the connectivity interface from the low-power state to the operational state based at least in part on determining that the first orientation does not satisfy the condition.

Clause 11: The system of Clause 10, wherein the analyte sensor electronics circuitry is further configured to: receive additional touch input data via the at least one input sensor; detect a second orientation of the analyte sensor electronics circuitry via the at least one input sensor; and based on determining that the second orientation satisfies the condition, discard the additional touch input data.

Clause 12: The system of Clause 11, wherein the condition corresponds to a user of the system sitting or lying on the at least one input sensor.

Clause 13: A system for on-demand transmission of analyte data, the system comprising: an analyte sensor; and analyte sensor electronics circuitry comprising a connectivity interface, the analyte sensor electronics circuitry configured to: process one or more radiofrequency (RF) signals received from a display device via the connectivity interface to identify a first wakeup pattern; based on identifying the first wakeup pattern, transition the connectivity interface from a low-power state to an operational state; upon transitioning the connectivity interface to the operational state, transmit first analyte data to the display device; and transition the connectivity interface from the operational state to the low-power state after transmitting the first analyte data to the display device.

Clause 14: The system of Clause 13, wherein the analyte sensor electronics circuitry is further configured to determine whether the display device is within a threshold distance of the connectivity interface, and the analyte sensor electronics circuitry is configured to transition the connectivity interface from the low-power state to the operational state based at least in part on determining that the display device is within the threshold distance.

Clause 15: The system of any of Clauses 13 or 14, wherein the first wakeup pattern comprises an on/off keying (OOK) pattern generated by a connectivity interface of the display device.

Clause 16: The system of Clause 15, wherein the connectivity interface of the display device comprises a Bluetooth Low-Energy (BLE) module.

Clause 17: The system of any of Clauses 13-16, wherein an RF wakeup circuit in communication with the connectivity interface is configured to process the one or more RF signals received to identify the first wakeup pattern while the connectivity interface is in the low-power state.

Clause 18: The system of any of Clauses 13-17, wherein the analyte sensor electronics circuitry is further configured to: process one or more additional RF signals received via the connectivity interface to identify a second wakeup pattern; based on identifying the second wakeup pattern, transition the connectivity interface from the low-power state to the operational state; and upon transitioning the connectivity interface to the operational state, transmit second analyte data to the display device.

Clause 19: The system of Clause 18, wherein the first analyte data comprises a first analyte type that corresponds to the first wakeup pattern, and the second analyte data comprises a second analyte type that corresponds to the second wakeup pattern.

Clause 20: The system of any of Clauses 13-19, wherein the one or more RF signals are generated by the display device in response to a user pressing a virtual button in an application executing on the display device.

Clause 21: A system for on-demand transmission of analyte data, the system comprising: an analyte sensor; a display device; at least one input sensor; and analyte sensor electronics circuitry comprising a connectivity interface, the analyte sensor electronics circuitry configured to: process touch input data received via the at least one input sensor to identify a first touch input pattern; based on identifying the first touch input pattern, transition the connectivity interface from a low-power state to an operational state; upon transitioning the connectivity interface to the operational state, transmit first analyte data to the display device, thereby providing on-demand transmission of analyte data based on touch input in a manner that reduces overall power consumption of the analyte sensor electronics circuitry; and transition the connectivity interface from the operational state to the low-power state after transmitting the first analyte data to the display device.

Clause 22: The system of Clause 21, wherein the at least one input sensor comprises a force sensor, and the first touch input pattern comprises at least one long-press.

Clause 23: The system of Clauses 21 or 22, wherein the at least one input sensor comprises an accelerometer and a force sensor, and the first touch input pattern comprises: (i) at least one tap detected via the accelerometer, and (ii) at least one long-press detected via the force sensor.

Clause 24: The system of any of Clauses 21-23, wherein the analyte sensor electronics circuitry acquires the analyte data via the analyte sensor while the connectivity interface is in the low-power state.

Clause 25: The system of any of Clauses 21-24, wherein the analyte sensor electronics circuitry is further configured to: process additional touch input data received via the at least one input sensor to identify a second touch input pattern; based on identifying the second touch input pattern, transition the connectivity interface from the low-power state to the operational state; and upon transitioning the connectivity interface to the operational state, transmit second analyte data to the display device.

Clause 26: The system of any of Clauses 21-25, wherein the first analyte data comprises a first analyte type that corresponds to the first touch input pattern, and the second analyte data comprises a second analyte type that corresponds to the second touch input pattern.

Clause 27: A system for on-demand transmission of analyte data, the system comprising: an analyte sensor; a display device; and analyte sensor electronics circuitry comprising a connectivity interface, the analyte sensor electronics circuitry configured to: process one or more radiofrequency (RF) signals received from the display device via the connectivity interface to identify a first wakeup pattern; based on identifying the first wakeup pattern, transition the connectivity interface from a low-power state to an operational state; upon transitioning the connectivity interface to the operational state, transmit first analyte data to the display device, thereby providing on-demand transmission of analyte data based on RF signals in a manner that reduces overall power consumption of the analyte sensor electronics circuitry; and transition the connectivity interface from the operational state to the low-power state after transmitting the first analyte data to the display device.

Clause 28: The system of Clause 27, wherein the analyte sensor electronics circuitry is further configured to determine whether the display device is within a threshold distance of the connectivity interface, and the analyte sensor electronics circuitry is configured to transition the connectivity interface from the low-power state to the operational state based at least in part on determining that the display device is within the threshold distance.

Clause 29: The system of Clauses 27 or 28, wherein the first wakeup pattern comprises an on/off keying (OOK) pattern generated by a connectivity interface of the display device.

Clause 30. The system of any of Clauses 27-29, wherein the connectivity interface of the display device comprises a Bluetooth Low-Energy (BLE) module.

Clause 31: The system of any of Clauses 27-30, wherein an RF wakeup circuit in communication with the connectivity interface is configured to process the one or more RF signals received to identify the first wakeup pattern while the connectivity interface is in the low-power state.

Clause 32: The system of any of Clauses 27-31, wherein the analyte sensor electronics circuitry is further configured to: process one or more additional RF signals received via the connectivity interface to identify a second wakeup pattern; based on identifying the second wakeup pattern, transition the connectivity interface from the low-power state to the operational state; and upon transitioning the connectivity interface to the operational state, transmit second analyte data to the display device.

Clause 33: The system of any of Clauses 27-32, wherein the first analyte data comprises a first analyte type that corresponds to the first wakeup pattern, and the second analyte data comprises a second analyte type that corresponds to the second wakeup pattern.

Clause 34: A method for on-demand transmission of analyte data, the method comprising: processing touch input data received via at least one input sensor to identify a first touch input pattern; based on identifying the first touch input pattern, transitioning a connectivity interface of analyte sensor electronics circuitry from a low-power state to an operational state; upon transitioning the connectivity interface to the operational state, transmitting first analyte data to a display device; and transitioning the connectivity interface from the operational state to the low-power state after transmitting the first analyte data to the display device.

Clause 35: The method of Clause 34, wherein the at least one input sensor comprises a force sensor, and the first touch input pattern comprises at least one long-press.

Clause 36: The method of Clauses 34 or 35, wherein the at least one input sensor comprises an accelerometer and a force sensor, and the first touch input pattern comprises: (i) at least one tap detected via the accelerometer, and (ii) at least one long-press detected via the force sensor.

Clause 37: The method of any of Clauses 34-36, further comprising acquiring, by the analyte sensor electronics circuitry, the analyte data via an analyte sensor while the connectivity interface is in the low-power state.

Clause 38: The method of any of Clauses 34-37, further comprising: processing additional touch input data received via the at least one input sensor to identify a second touch input pattern; based on identifying the second touch input pattern, transitioning the connectivity interface from the low-power state to the operational state; and upon transitioning the connectivity interface to the operational state, transmitting second analyte data to the display device.

Clause 39: The method of any of Clauses 34-38, wherein the first analyte data comprises a first analyte type that corresponds to the first touch input pattern, and the second analyte data comprises a second analyte type that corresponds to the second touch input pattern.

Clause 40: A method for on-demand transmission of analyte data, the system comprising: processing one or more radiofrequency (RF) signals received from a display device via a connectivity interface of analyte sensor electronics circuitry to identify a first wakeup pattern; based on identifying the first wakeup pattern, transitioning the connectivity interface from a low-power state to an operational state; upon transitioning the connectivity interface to the operational state, transmitting first analyte data to the display device; and transitioning the connectivity interface from the operational state to the low-power state after transmitting the first analyte data to the display device.

Clause 41: The method of Clause 40, further comprising determining whether the display device is within a threshold distance of the connectivity interface, wherein transitioning the connectivity interface from the low-power state to the operational state is based at least in part on determining that the display device is within the threshold distance.

Clause 42: The method of Clauses 40 or 41, wherein the first wakeup pattern comprises an on/off keying (OOK) pattern generated by a connectivity interface of the display device.

Clause 43: The method of any of Clauses 40-42, wherein the connectivity interface of the display device comprises a Bluetooth Low-Energy (BLE) module.

Clause 44: The method of any of Clauses 40-43, wherein processing the one or more RF signals is performed by an RF wakeup circuit in communication with the connectivity interface while the connectivity interface is in the low-power state.

Clause 45: The method of any of Clauses 40-44, further comprising: processing one or more additional RF signals received via the connectivity interface to identify a second wakeup pattern; based on identifying the second wakeup pattern, transitioning the connectivity interface from the low-power state to the operational state; and upon transitioning the connectivity interface to the operational state, transmitting second analyte data to the display device.

Clause 46: The method of any of Clauses 40-45, wherein the first analyte data comprises a first analyte type that corresponds to the first wakeup pattern, and the second analyte data comprises a second analyte type that corresponds to the second wakeup pattern.

### ADDITIONAL CONSIDERATIONS

Each of these non-limiting examples can stand on its own or can be combined in various permutations or combinations with one or more of the other examples. The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round”, a component that is not precisely circular (e.g., one that is slightly oblong or is a many-sided polygon) is still encompassed by this description.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A system for on-demand transmission of analyte data, the system comprising:
an analyte sensor;
at least one input sensor; and
analyte sensor electronics circuitry comprising a connectivity interface, the analyte sensor electronics circuitry configured to:
process touch input data received via the at least one input sensor to identify a first touch input pattern;
based on identifying the first touch input pattern, transition the connectivity interface from a low-power state to an operational state;
upon transitioning the connectivity interface to the operational state, transmit first analyte data to a display device; and
transition the connectivity interface from the operational state to the low-power state after transmitting the first analyte data to the display device.

2. The system of claim 1, wherein the at least one input sensor comprises a force sensor, and the first touch input pattern comprises at least one long-press.

3. The system of claim 1, wherein the at least one input sensor comprises an accelerometer and a force sensor, and the first touch input pattern comprises: (i) at least one tap detected via the accelerometer, and (ii) at least one long-press detected via the force sensor.

4. The system of claim 1, wherein the analyte sensor electronics circuitry acquires the analyte data via the analyte sensor while the connectivity interface is in the low-power state.

5. The system of claim 1, wherein the analyte sensor electronics circuitry is further configured to:
process additional touch input data received via the at least one input sensor to identify a second touch input pattern;
based on identifying the second touch input pattern, transition the connectivity interface from the low-power state to the operational state; and
upon transitioning the connectivity interface to the operational state, transmit second analyte data to the display device.

6. The system of claim 5, wherein the first analyte data comprises a first analyte type that corresponds to the first touch input pattern, and the second analyte data comprises a second analyte type that corresponds to the second touch input pattern.

7. The system of claim 1, wherein the analyte sensor electronics circuitry is further configured to:
receive additional touch input data via the at least one input sensor; and
in response to determining that a timeout period is active, discard the additional touch input data.

8. The system of claim 7, wherein the analyte sensor electronics circuitry is further configured to:
determine that touch input has been received a threshold number of times within a predetermined period of time, or
determine that the connectivity interface has been transitioned from the low-power state to the operational state the threshold number of times within the predetermined period of time; and
initiate the timeout period.

9. The system of claim 1, wherein the analyte sensor electronics circuitry is further configured to, prior to processing the touch input data:
transition a processor included in the analyte sensor electronics circuitry from a second low-power state to a second operational state in response to receiving initial touch input data via the at least one input sensor; and
determine that the touch input data was received within a threshold period of time of the initial touch input data,
wherein the analyte sensor electronics circuitry is configured to transition the connectivity interface from the low-power state to the operational state based at least in part on determining that the touch input data was received within the threshold period of time.

10. The system of claim 1, wherein the analyte sensor electronics circuitry is further configured to:
detect a first orientation of the analyte sensor electronics circuitry via the at least one input sensor; and
determine that the first orientation does not satisfy a condition,
wherein the analyte sensor electronics circuitry is configured to transition the connectivity interface from the low-power state to the operational state based at least in part on determining that the first orientation does not satisfy the condition.

11. The system of claim 10, wherein the analyte sensor electronics circuitry is further configured to:
receive additional touch input data via the at least one input sensor;
detect a second orientation of the analyte sensor electronics circuitry via the at least one input sensor; and
based on determining that the second orientation satisfies the condition, discard the additional touch input data.

12. The system of claim 11, wherein the condition corresponds to a user of the system sitting or lying on the at least one input sensor.

13. A system for on-demand transmission of analyte data, the system comprising:
an analyte sensor; and
analyte sensor electronics circuitry comprising a connectivity interface, the analyte sensor electronics circuitry configured to:
process one or more radiofrequency (RF) signals received from a display device via the connectivity interface to identify a first wakeup pattern;
based on identifying the first wakeup pattern, transition the connectivity interface from a low-power state to an operational state;
upon transitioning the connectivity interface to the operational state, transmit first analyte data to the display device; and
transition the connectivity interface from the operational state to the low-power state after transmitting the first analyte data to the display device.

14. The system of claim 13, wherein the analyte sensor electronics circuitry is further configured to determine whether the display device is within a threshold distance of the connectivity interface, and the analyte sensor electronics circuitry is configured to transition the connectivity interface from the low-power state to the operational state based at least in part on determining that the display device is within the threshold distance.

15. The system of claim 13, wherein the first wakeup pattern comprises an on/off keying (OOK) pattern generated by a connectivity interface of the display device.

16. The system of claim 15, wherein the connectivity interface of the display device comprises a Bluetooth Low-Energy (BLE) module.

17. The system of claim 13, wherein an RF wakeup circuit in communication with the connectivity interface is configured to process the one or more RF signals received to identify the first wakeup pattern while the connectivity interface is in the low-power state.

18. The system of claim 13, wherein the analyte sensor electronics circuitry is further configured to:
process one or more additional RF signals received via the connectivity interface to identify a second wakeup pattern;
based on identifying the second wakeup pattern, transition the connectivity interface from the low-power state to the operational state; and
upon transitioning the connectivity interface to the operational state, transmit second analyte data to the display device.

19. The system of claim 18, wherein the first analyte data comprises a first analyte type that corresponds to the first wakeup pattern, and the second analyte data comprises a second analyte type that corresponds to the second wakeup pattern.

20. The system of claim 13, wherein the one or more RF signals are generated by the display device in response to a user pressing a virtual button in an application executing on the display device.

21-46. (canceled)
